(19) United States
(12) Patent Application Publication
(10) Pub. No.: US 2022/0372559 A1
(43) Pub. Date: Nov. 24, 2022

(54) SYSTEM

(71) Applicant: Sherlock Biosciences, Inc., Brighton, MA (US)

(72) Inventors: William J. Blake, Winchester, MA (US); Carl Wayne Brown, III, Brighton, MA (US); Paul David Carlson, Brighton, MA (US)

(21) Appl. No.: 17/640,653

(22) PCT Filed: Sep. 9, 2020
(86) PCT No.: PCT/US2020/049961

§ 371 Date: Mar. 4, 2022

Related U.S. Application Data
(60) Provisional application No. 62/897,960, filed on Sep. 9, 2019.

(51) Int. Cl. C12Q 1/6816; G01N 33/52

(52) U.S. Cl. CPC C12Q 1/6816; G01N 33/521; C12Q 1/689

(57) ABSTRACT

The present disclosure describes technologies that permit sensitive detection of nucleic acids of interest (i.e., nucleic acids whose nucleotide sequence is or includes a target sequence). Among other things the disclosure provides a system comprising: a plurality of nucleic acid molecules having different nucleotide sequences; a set of ligation oligonucleotides, comprising: a first ligation oligonucleotide whose nucleotide sequence includes a templating element and a first target hybridization element; and a second ligation oligonucleotide whose nucleotide sequence includes a second target hybridization element and optionally a second templating element; wherein the target hybridization elements bind to different portions of a common target site, to form a gapped nucleic acid strand susceptible to ligation with a ligase to generate a ligated strand that is amenable to lateral flow assessment.

### CROSS REFERENCE TO RELATED APPLICATIONS

This application claims priority to U.S. Provisional Application No. 62/897,960 filed on Sep. 9, 2019, the contents of which is incorporated herein by reference in its entirety.

### BACKGROUND

It is increasingly important that technologies be developed that can specifically detect nucleic acids of interest, particularly when present in very low abundance.

### SUMMARY

The present disclosure describes technologies that permit sensitive detection of nucleic acids of interest (i.e., nucleic acids whose nucleotide sequence is or includes a target sequence), particularly through use of systems that achieve production of a detectable output (e.g., catalytic or non-catalytic output) when a target nucleic acid of interest is detected. Among other things, the present disclosure provides certain embodiments and formats for such systems that utilize or incorporate lateral flow technologies (e.g., for detection of one or more products that embody or represent [e.g., that are or are generated by] such output).

Advantages of certain embodiments of provided technologies may include, among other things, that (i) combining ligation-based detection with lateral flow assessment can facilitate point-of-care/service implementation and/or multiplexed detection embodiments; (ii) pre-amplification of target nucleic acid may not be required, thereby removing a potential source of sequence mutation that could distort assay results (e.g., via generating false positive and/or false negative read-outs); (iii) ligation specificity can enable single base discrimination; etc.

### BRIEF DESCRIPTION OF THE DRAWING

FIGS. 1-7 depict particular embodiments of technologies described herein.

FIGS. 8A and 8B demonstrate lateral flow test strips can be used as assay readout. The INSPECTR reaction was used to detect a short, synthetic RNA target (red). Cell-free protein synthesis (CFPS) of the resulting DNA expression cassette generated peptide antigen molecules (p24), which in turn can be visualized using a lateral flow test against p24. Only in the presence of the RNA target is a positive test line generated.

FIGS. 9A and 9B demonstrate multiple target regions can be detected by INSPECTR coupled to lateral flow readout. Different regions of the same RNA target (Influenza A region 1 and region 2) were detected using the same peptide antigen output.

FIGS. 10A and 10B demonstrate that fragmented peptide antigens are detectable on lateral flow. Fragments of the full length peptide antigen (p24A-F) containing putative antibody-binding domains were expressed in a cell-free protein synthesis reaction, starting from a double-stranded DNA expression cassette. All truncations were detectable on lateral flow above the no template control (Blank).

FIG. 11 demonstrates shorter peptide antigens enable rapid signal generation. Three peptide antigen outputs of varying lengths (245, 72, and 58 amino acids, respectively) were expressed from DNA expression cassettes and detected by lateral flow (LF) at reaction times varying from 0-4 h. Shorter peptide antigens can be expressed at a faster rate (molecules per time), as evidence by faster accumulation of strong lateral flow test bands. This rapid signal accumulation enables shorter reaction times to achieve the same output signal intensity.

### DEFINITIONS

[symbol] : in its broadest sense, as used herein, refers to any compound and/or substance that can be incorporated into a polypeptide chain, e.g., through formation of one or more peptide bonds. In some embodiments, an amino acid has the general structure H₂N—C(H)(R)—COOH. In some embodiments, an amino acid is a naturally-occurring amino acid. In some embodiments, an amino acid is a non-natural amino acid; in some embodiments, an amino acid is a D-amino acid; in some embodiments, an amino acid is an L-amino acid. “Standard amino acid” refers to any of the twenty standard L-amino acids commonly found in naturally occurring peptides. “Nonstandard amino acid” refers to any amino acid, other than the standard amino acids, regardless of whether it is prepared synthetically or obtained from a natural source. In some embodiments, an amino acid, including a carboxy- and/or amino-terminal amino acid in a polypeptide, can contain a structural modification as compared with the general structure above. For example, in some embodiments, an amino acid may be modified by methylation, amidation, acetylation, pegylation, glycosylation, phosphorylation, and/or substitution (e.g., of the amino group, the carboxylic acid group, one or more protons, and/or the hydroxyl group) as compared with the general structure. In some embodiments, such modification may, for example, alter the circulating half-life of a polypeptide containing the modified amino acid as compared with one containing an otherwise identical unmodified amino acid. In some embodiments, such modification does not significantly alter a relevant activity of a polypeptide containing the modified amino acid, as compared with one containing an otherwise identical unmodified amino acid. As will be clear from context, in some embodiments, the term “amino acid” may be used to refer to a free amino acid; in some embodiments it may be used to refer to an amino acid residue of a polypeptide.

[symbol] : As used herein, the term “analog” refers to a substance that shares one or more particular structural features, elements, components, or moieties with a reference substance. Typically, an “analog” shows significant structural similarity with the reference substance, for example sharing a core or consensus structure, but also differs in certain discrete ways. In some embodiments, an analog is a substance that can be generated from the reference substance, e.g., by chemical manipulation of the reference substance. In some embodiments, an analog is a substance that can be generated through performance of a synthetic process substantially similar to (e.g., sharing a plurality of steps with) one that generates the reference substance. In some embodiments, an analog is or can be generated through performance of a synthetic process different from that used to generate the reference substance.

[symbol] : Two events or entities are “associated” with one another, as that term is used herein, if the presence, level, degree, type and/or form of one is correlated with that of the other. For example, a particular entity (e.g., polypeptide, genetic signature, metabolite, microbe, etc) is considered to be associated with a particular disease, disorder, or condition, if its presence, level and/or form correlates with incidence of and/or susceptibility to the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

[symbol] : As used herein, the term “biological sample” typically refers to a sample obtained or derived from a biological source (e.g., a tissue or organism or cell culture) of interest, as described herein. In some embodiments, a source of interest is or comprises an organism, such as an animal or human. In some embodiments, a biological sample is or comprises biological tissue or fluid. In some embodiments, a biological sample may be or comprise bone marrow; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as a ductal lavages or broncheoalveolar lavages; aspirates; scrapings; bone marrow specimens; tissue biopsy specimens; surgical specimens; feces, other body fluids, secretions, and/or excretions; and/or cells therefrom, etc. In some embodiments, a biological sample is or comprises cells obtained from an individual. In some embodiments, obtained cells are or include cells from an individual from whom the sample is obtained. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, collection of body fluid (e.g., blood, lymph, feces etc.), etc. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to techniques such as amplification or reverse transcription of mRNA, isolation and/or purification of certain components, etc.

[symbol] : As used herein, the term “characteristic portion”, in the broadest sense, refers to a portion of a substance whose presence (or absence) correlates with presence (or absence) of a particular feature, attribute, or activity of the substance. In some embodiments, a characteristic portion of a substance is a portion that is found in the substance and in related substances that share the particular feature, attribute or activity, but not in those that do not share the particular feature, attribute or activity. In certain embodiments, a characteristic portion shares at least one functional characteristic with the intact substance. For example, in some embodiments, a “characteristic portion” of a protein or polypeptide is one that contains a continuous stretch of amino acids, or a collection of continuous stretches of amino acids, that together are characteristic of a protein or polypeptide. In some embodiments, each such continuous stretch generally contains at least 2, 5, 10, 15, 20, 50, or more amino acids. In general, a characteristic portion of a substance (e.g., of a protein, antibody, etc.) is one that, in addition to the sequence and/or structural identity specified above, shares at least one functional characteristic with the relevant intact substance. In some embodiments, a characteristic portion may be biologically active.

[symbol] : A “characteristic sequence” is a sequence that is found in all members of a family of polypeptides or nucleic acids, and therefore can be used by those of ordinary skill in the art to define members of the family.

[symbol] : As used herein, the phrase “characteristic sequence element” refers to a sequence element found in a polymer (e.g., in a polypeptide or nucleic acid) that represents a characteristic portion of that polymer. In some embodiments, presence of a characteristic sequence element correlates with presence or level of a particular activity or property of the polymer. In some embodiments, presence (or absence) of a characteristic sequence element defines a particular polymer as a member (or not a member) of a particular family or group of such polymers. A characteristic sequence element typically comprises at least two monomers (e.g., amino acids or nucleotides). In some embodiments, a characteristic sequence element includes at least 2, 3, 4, 5, 6, 7, 8, 9, 10, 11, 12, 13, 14, 15, 20, 25, 30, 35, 40, 45, 50, or more monomers (e.g., contiguously linked monomers). In some embodiments, a characteristic sequence element includes at least first and second stretches of contiguous monomers spaced apart by one or more spacer regions whose length may or may not vary across polymers that share the sequence element.

[symbol] : As use herein, the term “comparable” refers to two or more agents, entities, situations, sets of conditions, etc., that may not be identical to one another but that are sufficiently similar to permit comparison there between so that one skilled in the art will appreciate that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

[symbol] : As used herein, the term “corresponding to” may be used to designate the position/identity of a structural element in a compound or composition through comparison with an appropriate reference compound or composition. For example, in some embodiments, a monomeric residue in a polymer (e.g., an amino acid residue in a polypeptide or a nucleic acid residue in a polynucleotide) may be identified as “corresponding to” a residue in an appropriate reference polymer. For example, those of ordinary skill will appreciate that, for purposes of simplicity, residues in a polypeptide are often designated using a canonical numbering system based on a reference related polypeptide, so that an amino acid “corresponding to” a residue at position 190, for example, need not actually be the 190^{th} amino acid in a particular amino acid chain but rather corresponds to the residue found at 190 in the reference polypeptide; those of ordinary skill in the art readily appreciate how to identify “corresponding” amino acids. For example, those skilled in the art will be aware of various sequence alignment strategies, including software programs such as, for example, BLAST, CS-BLAST, CUSASW++, DIAMOND, FASTA, GGSEARCH/GLSEARCH, Genoogle, HMMER, HHpred/HHsearch, IDF, Infernal, KLAST, USEARCH, parasail, PSI-BLAST, PSI-Search, ScalaBLAST, Sequilab, SAM, SSEARCH, SWAPHI, SWAPHI-LS, SWIMM, or SWIPE that can be utilized, for example, to identify “corresponding” residues in polypeptides and/or nucleic acids in accordance with the present disclosure.

[symbol] : The term “detectable entity” as used herein refers to any element, molecule, functional group, compound, fragment or moiety that is detectable. In some embodiments, a detectable entity is provided or utilized alone. In some embodiments, a detectable entity is provided and/or utilized in association with (e.g., joined to) another agent. Examples of detectable entities include, but are not limited to: various ligands, radionuclides (e.g., ³H, ¹⁴C, ¹⁸F, ¹⁹F, ³²P, ³⁵S, ¹³⁵I, ¹²⁵I, ¹²³I, ⁶⁴Cu, ¹⁸⁷Re, ¹¹¹In, ⁹⁰Y, ^{99m}Tc, ¹⁷⁷Lu, ⁸⁹Zr etc.), fluorescent dyes (for specific exemplary fluorescent dyes, see below), chemiluminescent agents (such as, for example, acridinum esters, stabilized dioxetanes, and the like), bioluminescent agents, spectrally resolvable inorganic fluorescent semiconductors nanocrystals (i.e., quantum dots), metal nanoparticles (e.g., gold, silver, copper, platinum, etc.) nanoclusters, paramagnetic metal ions, enzymes (for specific examples of enzymes, see below), colorimetric labels (such as, for example, dyes, colloidal gold, and the like), biotin, dioxigenin, haptens, and proteins for which antisera or monoclonal antibodies are available.

[symbol] : In general, the term “engineered” refers to the aspect of having been manipulated by the hand of man. For example, a polynucleotide is considered to be “engineered” when two or more sequences, that are not linked together in that order in nature, are manipulated by the hand of man to be directly linked to one another in the engineered polynucleotide. For example, in some embodiments of the present invention, an engineered polynucleotide comprises a regulatory sequence that is found in nature in operative association with a first coding sequence but not in operative association with a second coding sequence, is linked by the hand of man so that it is operatively associated with the second coding sequence. Comparably, a cell or organism is considered to be “engineered” if it has been manipulated so that its genetic information is altered (e.g., new genetic material not previously present has been introduced, for example by transformation, mating, somatic hybridization, transfection, transduction, or other mechanism, or previously present genetic material is altered or removed, for example by substitution or deletion mutation, or by mating protocols). As is common practice and is understood by those in the art, progeny of an engineered polynucleotide or cell are typically still referred to as “engineered” even though the actual manipulation was performed on a prior entity.

[symbol] : A marker, as used herein, refers to an entity or moiety whose presence or level is a characteristic of a particular state or event. In some embodiments, presence or level of a particular marker may be characteristic of presence or stage of a disease, disorder, or condition. To give but one example, in some embodiments, the term refers to a gene expression product that is characteristic of a particular tumor, tumor subclass, stage of tumor, etc. Alternatively or additionally, in some embodiments, a presence or level of a particular marker correlates with activity (or activity level) of a particular signaling pathway, for example that may be characteristic of a particular class of tumors. The statistical significance of the presence or absence of a marker may vary depending upon the particular marker. In some embodiments, detection of a marker is highly specific in that it reflects a high probability that the tumor is of a particular subclass. Such specificity may come at the cost of sensitivity (i.e., a negative result may occur even if the tumor is a tumor that would be expected to express the marker). Conversely, markers with a high degree of sensitivity may be less specific that those with lower sensitivity. According to the present invention a useful marker need not distinguish tumors of a particular subclass with 100% accuracy.

[symbol] : As used herein, in its broadest sense, refers to any compound and/or substance that is or can be incorporated into an oligonucleotide chain. In some embodiments, a nucleic acid is a compound and/or substance that is or can be incorporated into an oligonucleotide chain via a phosphodiester linkage. As will be clear from context, in some embodiments, “nucleic acid” refers to an individual nucleic acid residue (e.g., a nucleotide and/or nucleoside); in some embodiments, “nucleic acid” refers to an oligonucleotide chain comprising individual nucleic acid residues. In some embodiments, a “nucleic acid” is or comprises RNA; in some embodiments, a “nucleic acid” is or comprises DNA. In some embodiments, a nucleic acid is, comprises, or consists of one or more natural nucleic acid residues. In some embodiments, a nucleic acid is, comprises, or consists of one or more nucleic acid analogs. In some embodiments, a nucleic acid analog differs from a nucleic acid in that it does not utilize a phosphodiester backbone. For example, in some embodiments, a nucleic acid is, comprises, or consists of one or more “peptide nucleic acids”, which are known in the art and have peptide bonds instead of phosphodiester bonds in the backbone, are considered within the scope of the present invention. Alternatively or additionally, in some embodiments, a nucleic acid has one or more phosphorothioate and/or 5′-N-phosphoramidite linkages rather than phosphodiester bonds. In some embodiments, a nucleic acid is, comprises, or consists of one or more natural nucleosides (e.g., adenosine, thymidine, guanosine, cytidine, uridine, deoxyadenosine, deoxythymidine, deoxy guanosine, and deoxycytidine). In some embodiments, a nucleic acid is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, and combinations thereof). In some embodiments, a nucleic acid comprises one or more modified sugars (e.g., 2′-fluororibose, ribose, 2′-deoxyribose, arabinose, and hexose) as compared with those in natural nucleic acids. In some embodiments, a nucleic acid has a nucleotide sequence that encodes a functional gene product such as an RNA or protein. In some embodiments, a nucleic acid includes one or more introns. In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, a nucleic acid is partly or wholly single stranded; in some embodiments, a nucleic acid is partly or wholly double stranded. In some embodiments a nucleic acid has a nucleotide sequence comprising at least one element that encodes, or is the complement of a sequence that encodes, a polypeptide. In some embodiments, a nucleic acid has enzymatic activity. Those skilled in the art, reading the present specification, will appreciate that ligation oligonucleotide sets, activating nucleic acids, and/or guide RNAs can each be engineered and/or manipulated, e.g., to incorporate nucleotide analogs, etc.

[symbol] : as used herein, refers to a juxtaposition wherein the components described are in a relationship permitting them to function in their intended manner. A control element “operably linked” to a functional element is associated in such a way that expression and/or activity of the functional element is achieved under conditions compatible with the control element. In some embodiments, “operably linked” control elements are contiguous (e.g., covalently linked) with the coding elements of interest; in some embodiments, control elements act in trans to or otherwise at a from the functional element of interest.

[symbol] : As used herein refers to any polymeric chain of amino acids. In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man. In some embodiments, a polypeptide may comprise or consist of natural amino acids, non-natural amino acids, or both. In some embodiments, a polypeptide may comprise or consist of only natural amino acids or only non-natural amino acids. In some embodiments, a polypeptide may comprise D-amino acids, L-amino acids, or both. In some embodiments, a polypeptide may comprise only D-amino acids. In some embodiments, a polypeptide may comprise only L-amino acids. In some embodiments, a polypeptide may include one or more pendant groups or other modifications, e.g., modifying or attached to one or more amino acid side chains, at the polypeptide's N-terminus, at the polypeptide's C-terminus, or any combination thereof. In some embodiments, such pendant groups or modifications may be selected from the group consisting of acetylation, amidation, lipidation, methylation, pegylation, etc., including combinations thereof. In some embodiments, a polypeptide may be cyclic, and/or may comprise a cyclic portion. In some embodiments, a polypeptide is not cyclic and/or does not comprise any cyclic portion. In some embodiments, a polypeptide is linear. In some embodiments, a polypeptide may be or comprise a stapled polypeptide. In some embodiments, the term “polypeptide” may be appended to a name of a reference polypeptide, activity, or structure; in such instances it is used herein to refer to polypeptides that share the relevant activity or structure and thus can be considered to be members of the same class or family of polypeptides. For each such class, the present specification provides and/or those skilled in the art will be aware of exemplary polypeptides within the class whose amino acid sequences and/or functions are known; in some embodiments, such exemplary polypeptides are reference polypeptides for the polypeptide class or family. In some embodiments, a member of a polypeptide class or family shows significant sequence homology or identity with, shares a common sequence motif (e.g., a characteristic sequence element) with, and/or shares a common activity (in some embodiments at a comparable level or within a designated range) with a reference polypeptide of the class; in some embodiments with all polypeptides within the class). For example, in some embodiments, a member polypeptide shows an overall degree of sequence homology or identity with a reference polypeptide that is at least about 30-40%, and is often greater than about 50%, 60%, 70%, 80%, 90%, 91%, 92%, 93%, 94%, 95%, 96%, 97%, 98%, 99% or more and/or includes at least one region (e.g., a conserved region that may in some embodiments be or comprise a characteristic sequence element) that shows very high sequence identity, often greater than 90% or even 95%, 96%, 97%, 98%, or 99%. Such a conserved region usually encompasses at least 3-4 and often up to 20 or more amino acids; in some embodiments, a conserved region encompasses at least one stretch of at least 2, 3, 4, 5, 6, 7, 8, 9, 10, 11, 12, 13, 14, 15 or more contiguous amino acids. In some embodiments, a relevant polypeptide may comprise or consist of a fragment of a parent polypeptide. In some embodiments, a useful polypeptide as may comprise or consist of a plurality of fragments, each of which is found in the same parent polypeptide in a different spatial arrangement relative to one another than is found in the polypeptide of interest (e.g., fragments that are directly linked in the parent may be spatially separated in the polypeptide of interest or vice versa, and/or fragments may be present in a different order in the polypeptide of interest than in the parent), so that the polypeptide of interest is a derivative of its parent polypeptide.

[symbol] : As used herein describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

[symbol] : As used herein, the term “specific binding” refers to an ability to discriminate between possible binding partners in the environment in which binding is to occur. A binding agent that interacts with one particular target when other potential targets are present is said to “bind specifically” to the target with which it interacts. In some embodiments, specific binding is assessed by detecting or determining degree of association between the binding agent and its partner; in some embodiments, specific binding is assessed by detecting or determining degree of dissociation of a binding agent-partner complex; in some embodiments, specific binding is assessed by detecting or determining ability of the binding agent to compete an alternative interaction between its partner and another entity. In some embodiments, specific binding is assessed by performing such detections or determinations across a range of concentrations.

### DETAILED DESCRIPTION OF CERTAIN EMBODIMENTS

In various embodiments, the present disclosure provides systems that utilize ligation-based technologies for initial (and target-sequence dependent) hybridization to target nucleic acids of interest, and uses those systems to generate a detectable output

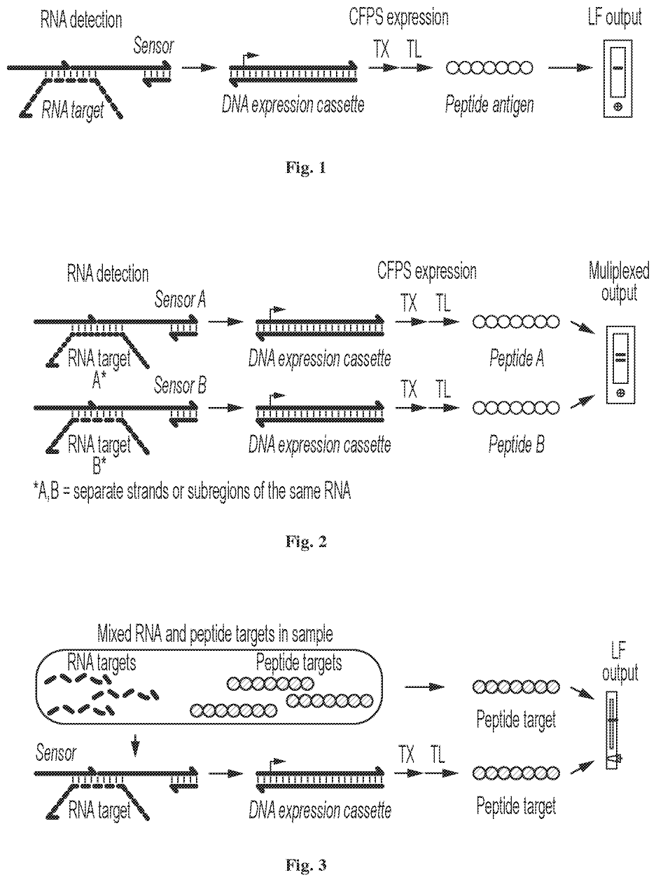

FIG. 1 depicts an exemplary embodiment of nucleic acid detection in accordance with the present disclosure. As indicated in FIG. 1, a nucleic acid sample that may include at least one nucleic acid of interest (i.e., whose nucleotide sequence is or includes a target site of interest) is contacted with a set of ligation oligonucleotides, which set comprises (i) a first ligation oligonucleotide whose nucleotide sequence includes a first templating element and a first target hybridization element; (ii) a second ligation oligonucleotide whose nucleotide sequence includes a second target hybridization element and a second templating element; and (iii) optionally one or more bridging oligonucleotides whose nucleotide sequence is or comprises one or more additional target hybridization elements. The oligonucleotides of the set are selected so that, when they are simultaneously hybridized to the nucleic acid of interest, they abut one another so that a ligase enzyme can link them to form a single ligated strand. The ligated strand is or is a complement or component (e.g., is one strand of a double-stranded entity) of a nucleic acid that, for example can be expressed (e.g., transcribed and/or translated) to generate a detectable output.

In some embodiments, as depicted in FIG. 1, the ligated strand is copied by a system that acts on the templating element. For example, where a templating element is or comprises a promoter (or its complement) and/or one or more transcriptional regulatory elements (or complements thereof), the system may be or comprise an RNA polymerase; where a templating element is or comprises an origin of replication and/or a binding site (or complement thereof) for an extendible primer, the system may be or comprise a DNA polymerase (which, in some embodiments, may be a thermostable DNA polymerase, particularly if the ligated strand includes a sequence element corresponding to a second extendible primer and the system includes an appropriate pair of primers to amplify a duplex of the ligated strand and its complement). In some embodiments, as demonstrated in FIG. 1, ligation of a first and second ligation oligonucleotide results in a nucleic acid template whose expression or other copying can achieve a detectable output as described herein. In some embodiments the ligated strand (or its complement) is transcribed and/or translated (e.g., via cell-free components such as a cell free protein synthesis expression system (CFPS)).

In some embodiments, ligation as described herein generates a detectable output. In some embodiments, such detectable output is or is generated by a polypeptide. In some embodiments, a detectable output may be or comprise a catalytic output; in some embodiments, a detectable output may be or comprise a non-catalytic output.

In some embodiments, a catalytic output is or is generated by an enzyme that catalyzes a reaction, e.g., converting one or more substrates to one or more detectable. In some embodiments, a non-catalytic output is or generates a detectable nucleic acid or polypeptide (e.g., that act as an antigen or other specific binding ligand).

Among other things, the present disclosure provides technology formats in which a detectable output is amenable to lateral flow analysis (e.g., is, comprises, or generates a product that is detectable by lateral flow). In some embodiments, the present disclosure provides an insight that coupling ligation mediated detection technologies with lateral flow assessment technologies may particularly facilitate multiplexed analyses (e.g., simultaneous detection of a plurality of products amenable to lateral flow). Furthermore, the present disclosure teaches that such coupling may have particular advantages that permit effective multiplexed analysis of products of different chemical class (e.g., two or more of nucleic acids, metals, polypeptides, small molecules, antibodies or fragments thereof etc.).

Below, certain features and/or attributes of various elements and/or embodiments of the present invention are discussed in more detail. Those of ordinary skill, reading the disclosure, will be aware of certain modifications and/or variations that are within the skill of one of ordinary skill and also are within the spirit and scope of this disclosure. Those of ordinary skill will also appreciate that, given the templating property of nucleic acid strands, and their ability to hybridize with one another, it is conventional in the field to refer to a particular “sequence element” or “site” and relying on the skilled artisan to understand from context when the “forward” or “sense” form is being referenced, and when its complement (the “reverse” or “antisense” form).

### Target Nucleic Acid

Those skilled in the art will immediately appreciate that technologies provided herein are broadly applicable to achieve detection of a wide range of nucleic acids including, for example, nucleic acids from an infectious agent (e.g., a virus, microbe, parasite, etc), nucleic acids indicative of a particular physiological state or condition (e.g., presence or state of a disease, disorder or condition such as, for example, cancer or an inflammatory or metabolic disease, disorder or condition, etc), prenatal nucleic acids, etc.

In many embodiments, provided technologies are particularly useful or applicable for detection of low-abundance (e.g., less than about 10 fM, or about 1 fM, or about 100 aM) nucleic acids.

Typically, provided technologies will be applied to one or more samples to assess presence and/or level of one or more target nucleic acids in the sample. In some embodiments, the sample is a biological sample; in some embodiments, a sample is an environmental sample.

In some embodiments, a sample will be processed (e.g., nucleic acids will be partially or substantially isolated or purified out of a primary sample); in some embodiments, only minimal processing will have been performed (i.e., the sample will be a crude sample).

### Ligation Oligonucleotide Sets

As described herein, in some embodiments, the present disclosure utilizes ligation technologies for hybridization with/detection of initial target nucleic acids. The ligation step is therefore sequence-specific to the target sequence of interest; for each such target site, a set of ligation oligonucleotides is designed that together hybridize across a target sequence of interest, adjacent to one another so that activity of a ligase links hybridized oligonucleotides together to form a single ligated strand. This ligated strand includes the complement of the entire target site selected, and also includes a sensor element, and typically an expression element, that, prior to ligation, were not part of the same oligonucleotide.

Those skilled in the art will be aware of a variety of systems that utilize sets of ligation oligonucleotides to bring together functional elements located on separate oligonucleotides of the set only when the set becomes linked by ligation, as occurs in the presence of a target nucleic acid of interest, to which each of the oligonucleotides in the set hybridizes at adjacent portions of a target site, is present. (see, for example, promoter-ligation-activated-transcription amplification of nucleic acid sequences, as described in U.S. Pat. No. 5,194,370; multiplex ligatable probe amplification [MLPA], as described in U.S. Pat. No. 6,955,901, and Nucleic Acids Research 30:e27, 2002; RNA-splinted polynucleotide ligase activity as described, for example, in Nucleic Acids Research 42:1831, 2013, US patent application US2014/0179539 and Nucleic Acids Research 33:e116, 2016). Those of ordinary skill are therefore well aware of design parameters relevant to construction of oligonucleotides within oligonucleotide sets as described herein (see also US patent application US2008/0090238)

### Target Hybridization Elements

As depicted in FIG. 1, ligation oligonucleotide sets for use in accordance with the present disclosure are designed so that each includes a sequence element that hybridizes to a target nucleic acid at a position adjacent to that where another oligonucleotide of the set, such that when all oligonucleotides of a particular set are hybridized to a target nucleic acid (i.e., to a nucleic acid that includes a target site), they can be covalently linked to one another by a ligase.

In some embodiments, a set of ligation oligonucleotides includes only two (i.e., first and second) oligonucleotides, each of which includes a target hybridization element and one of which includes a primer element; in some embodiments, the other includes a templating element. In some embodiments, a set of ligation oligonucleotides includes one or more bridging oligonucleotides that hybridize to the target site between the first and second oligonucleotides.

Those skilled in the art, reading the present disclosure, will appreciate that the target site can be any sequence of interest—e.g., whose presence in a particular sample is to be assessed. Those skilled in the art will further be aware (e.g., from other ligation systems described in the literature, including those cited herein) of design considerations relevant to selecting length and/or sequence characteristics (e.g., GC content etc) of individual target hybridization elements in oligonucleotides within a set of ligation oligonucleotides.

### Templating Elements

At least one oligonucleotide within a ligation oligonucleotide set will typically include a templating element, which permits and/or directs templating of the ligated strand.

### Primer Binding Elements

In many embodiments, a templating element will be or comprise a binding site for an extendable oligonucleotide (i.e., a primer, so that the templating element is a “primer binding element”). Incubation of the ligated strand with such an oligonucleotide and an appropriate extending enzyme (e.g., a DNA polymerase) generates a template strand, so that a duplex nucleic acid is created.

In some embodiments, such a duplex nucleic acid includes a binding site for second extendable oligonucleotide (i.e., a second primer), so that the duplex can be amplified by polymerase chain reaction; in such embodiments, the binding site for the second extendable oligonucleotide (i.e., a second primer binding element) is typically in the second oligonucleotide, on the far end of the primer element, relative to the first oligonucleotide, so that when the duplex is incubated with the first and second primer (or a different primer pair) and a DNA polymerase (e.g., a thermostable DNA polymerase), multiple copies of a duplex whose strands span the ligation junction is generated.

Those skilled in the art, reading the present disclosure, will appreciate that a primer element is a sequence (or, in some embodiments, the complement thereof) that is bound by an extendible nucleic acid (e.g., oligonucleotide); such an extendible nucleic acid can be designed, selected, and/or optimized particularly for use in detection systems as described herein. As already noted, one feature of provided technologies is that it is not necessary to design or utilize a different primer element for each target nucleic acid; the primer element need not (and, typically, does not) hybridize to the target nucleic acid during the ligation step.

In some embodiments of the present disclosure, for example, where multiple ligation oligonucleotide sets (i.e., designed for detection of different target nucleic acids) are to be used simultaneously (e.g., together in the same reactions), it may be desirable that different ligation oligonucleotide sets have different primer elements (e.g., each ligation oligonucleotide set may have its own primer element), but in some embodiments, multiple different ligation oligonucleotide sets, or even all ligation oligonucleotide sets, may include the same primer element.

### Expression Elements

In some embodiments, a templating element may be or comprise a sequence (i.e., an “expression element”) that directs (or is the complement of one that directs) expression of associated sequences. For example, in some embodiments, an expression element is or comprises a promoter and/or one or more transcriptional regulatory elements (e.g., enhancers, repressor binding sites, etc), so that multiple RNA templates (or copies) of the ligated strand are generated by incubating, e.g., a duplex as described above, with an RNA polymerase.

In some embodiments, such an expression element may be or comprise an origin of replication, so that multiple DNA templates (or copies) of the ligated strand are generated by incubation with a DNA polymerase.

Those of ordinary skill in the art, reading the present disclosure, will appreciate that an appropriate expression element for use in a particular embodiment may be selected to generate (or permit ready generation of—e.g., by denaturation) a templated product (e.g., RNA, ssDNA, or dsDNA) that is effective to be or generate a detectable output.

For example, FIG. 1 depicts a system in which a first ligation oligonucleotide comprises an expression element (specifically, a promoter), and a first target hybridization element, and a second ligation oligonucleotide comprises a second target hybridization element and a primer element. Ligation of these two oligonucleotides generates a ligated strand that is copied by extension of a primer binding to the primer element (and, optionally, amplified, by extension of the same primer or a different primer together with a displaced primer that hybridizes to the opposing strand, as would be understood by those skilled in the art). Transcription from the expression element generates a transcript that is translated into a peptide antigen amenable to detection by lateral flow.

### Applications

Those skilled in the art, reading the present specification, will immediately appreciate that technology it provides is useful in a wide range of contexts, and can be applied in a variety of formats.

In some embodiments, one or more components (e.g., target nucleic acid, ligation oligonucleotide(s), ligated strand, primer(s), templating component(s) (e.g., DNA polymerase, RNA polymerase, nucleotides, etc), expression components (e.g., RNA polymerase, ribosome, nucleotides, tRNAs, etc), and combinations thereof) may be associated with (e.g., attached to) a solid support.

In some embodiments, a plurality of ligation oligonucleotide sets is utilized substantially simultaneously, so that multiple target nucleic acids may be detected contemporaneously.

In some embodiments, provided technologies may be multiplexed, for example. Indeed, as described herein, in some embodiments, provided technologies may be particularly useful for multiplexed (e.g., simultaneous) analyses of a plurality of products amenable to lateral flow assessment.

### Lateral Flow Assessment

Those skilled in the art, reading the present disclosure, will appreciate that a variety of lateral flow formats are available that can be used in accordance with the practice of the present invention. See, for example, US 2009/0203059; US 2009/0053738; U.S. Pat. Nos. 8,846,328; 6,673,628; 7,097,983; 8,535,617.

Lateral flow technologies can be particularly useful for nucleic acid detection systems as described herein at least because certain such technologies have been demonstrated to be robust and/or amenable to miniaturization sufficient to permit point-of-care/service formats (e.g., dipstick, portable device etc). Such formats may be particularly useful for certain embodiments of the present disclosure including, to name just a few, field or home detection of infectious agents.

In some embodiments, point-of-care/service formats may be particularly useful when it is desirable to minimize exposure (e.g., third party exposure) to a particular infectious agent. For example, a scanning team could assess an environmental site (e.g., a battlefield or other site) before permitting entry. Alternatively or additionally, a potential patient could avoid traveling to a medical site (e.g., a hospital or clinic), thereby minimizing his/her own exposure to others at that site and/or minimizing exposure of those at the site to his/her potential infection.

In some embodiments, provided technologies are utilized in a hospital context (e.g., at hospital admission, for example to assess whether quarantine might be appropriate). In some embodiments, provided technologies are utilized in a battlefield context. In some embodiments, provided technologies are utilized in a home setting.

In some embodiments, provided technologies are utilized to assess responsiveness to or success of a particular therapeutic regimen—e.g., to treat an infection and/or to reduce (or increase) presence of a particular nucleic acid in a subject (e.g., which nucleic acid may be associated with a particular disease, disorder or condition, or may be associated with a particular therapy).

### Exemplification

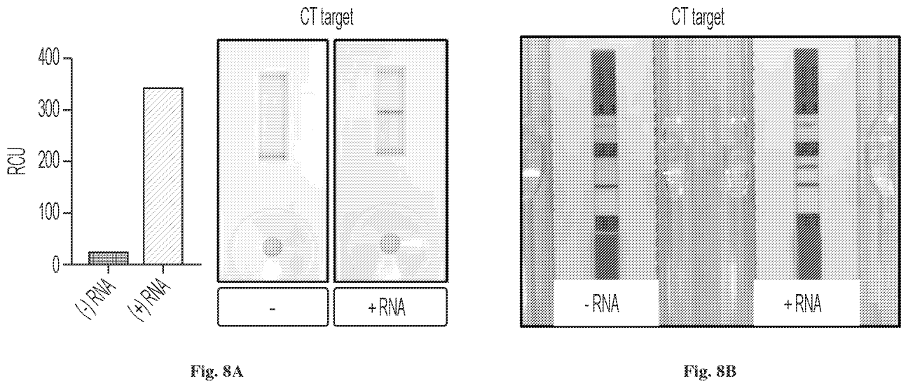

Example 1: As shown in FIG. 1, in some embodiments a system as described herein can detect a nucleic acid target in a sample. That detection can lead to production of a detectable output, e.g., a polypeptide antigen, which can be detected in a lateral flow device. FIGS. 8A and 8B confirm detection of a nucleic acid target in a sample by systems described herein. FIGS. 8A-8B specifically show the detection of Chlamydia (CT) target nucleic acid in sample by generation of p24 peptide antigen using prototype (FIG. 8A) and commercial (FIG. 8B) lateral flow test strips. Samples containing CT target nucleic acids were contacted with the system comprising a set of ligation oligonucleotides as described in the current application. The mixture was incubated at room temperature for 15 minutes in the presence of a ligase. The mixture was then contacted with a cell-free protein synthesis (CFPS) composition at room temperature for 2 hours to allow the transcription and translation of the target nucleic acids. The product of the reaction is loaded with a lateral flow strip. For controls, the lateral flow strip was load with a reaction product lacking the target nucleic acid. The presence of the target nucleic acid was detected by the appearance of a visible band on the lateral flow strip at the desired location. No band was detected on the control strips at the expected location. A quantification of the intensity of the bands (FIG. 8A) indicated that the intensity of the band generated by the detection of the target nucleic acids was significantly higher than that on the control strips.

Example 2: As shown in FIG. 2, in some embodiments a system as described herein can enable multiplexed detection. In some embodiments, two or more target nucleic acids activates expression of separate detectable outputs. In some embodiments, a single target nucleic acid activates two separate sets of ligation oligonucleotides each encoding a separate detectable output.

Example 3: As shown in FIG. 3, in some embodiments a system as described herein can be used in combined detection of protein and nucleic acid. As shown in FIG. 3, a system described herein can detect a peptide target occurring in a sample using lateral flow methods/chemistries. Alternatively, or additionally, a system described herein can detect the same peptide target as a detectable output generated by detection of an RNA target in the sample and ligation of oligonucleotide sets.

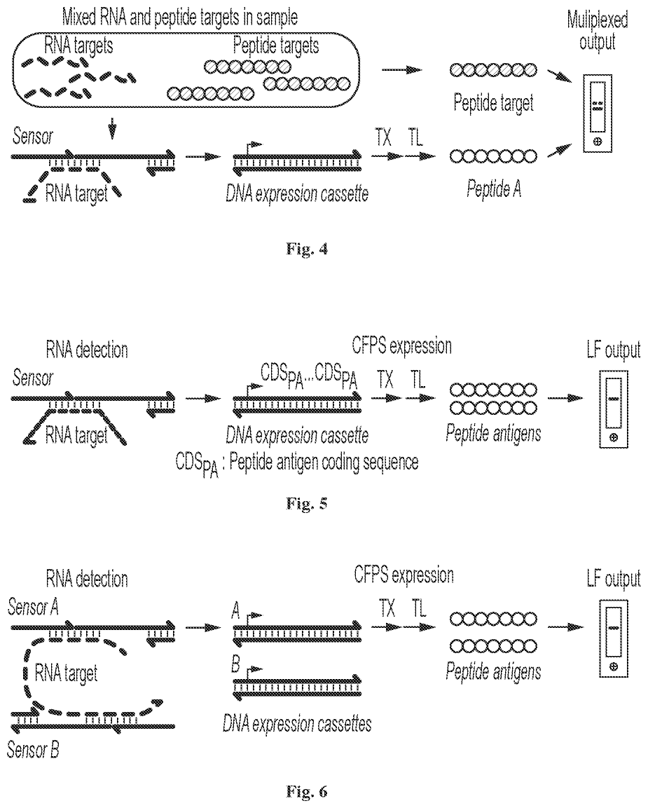

Example 4: As shown in FIG. 4, a system described herein can be used for separate detection of both a target nucleic acid and a peptide where the epitope on the peptide is detected via lateral flow-based methods/chemistries combined with production of separately detectable output epitope via by detection of an RNA target in the sample and ligation of oligonucleotide sets.

Example 5: As shown in FIG. 5, a system described herein can be used for generation of multiple detectable outputs based on the detection of a single RNA target in a sample. Detection of a target RNA in a sample ligating two oligonucleotide sets can result in a DNA expression cassette encoding concatenated detectable outputs.

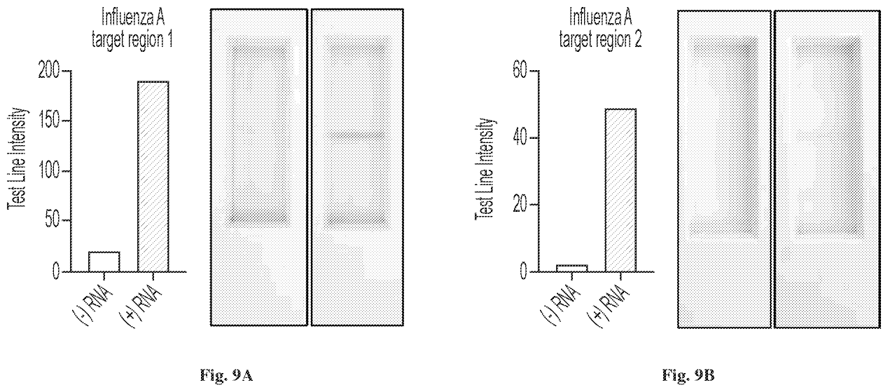

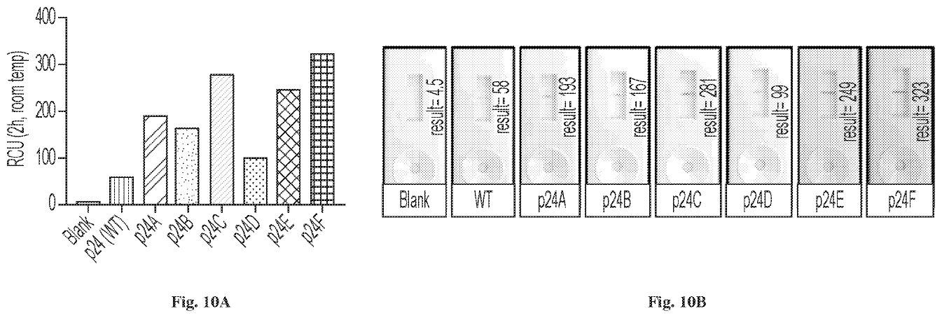

Example 6: As shown in FIG. 6, a system described herein can detect multiple regions in a single target nucleic acid. Two or more sets of ligation oligonucleotides can be ligated by two or more different regions of a target nucleic acid. Ligation of each oligonucleotide set can result in expression of a detectable output (e.g., a peptide antigen). Detection of multiple regions in a single target nucleic acid by systems described herein is confirmed by FIGS. 9A and 9B. FIGS. 9A and 9B specifically show the detection of influenza A target region 1 (FIG. 9A) and influenza A target region 2 (FIG. 9B) target nucleic acids in a sample. Samples containing influenza A were contacted with the system comprising a set of ligation oligonucleotides as described in the current application. The mixture was incubated at room temperature for 15 minutes in the presence of a ligase. The mixture was then contacted with a cell free composition at room temperature for 2 hours to allow the transcription and translation of the target nucleic acids. The product of the reaction was loaded with a lateral flow strip. For controls, the lateral flow strip was loaded with reaction products lacking the target nucleic acid. The presence of the target nucleic acid was detected by the appearance of a visible band on the lateral flow strip. No band was detected on the control strips. A quantification of the intensity of the bands indicated that the intensity of the band generated by the detection of the target nucleic acids is significantly higher than that on the control strips.

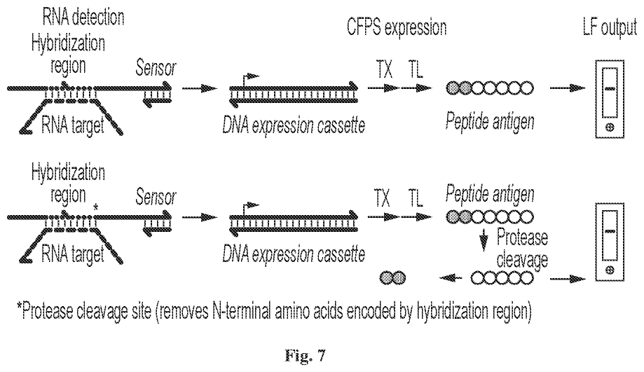

Example 7: As shown in FIG. 7, a system described herein can involve addition of a protease cleavage site between the hybridization region and the coding sequence in cases where the hybridization region is on the N-terminal region of the output protein. Thus, as shown in FIG. 7 a detectable output that is a peptide antigen can be detected via lateral flow-based methods/chemistries or a detectable output that is a peptide antigen can be cleaved by a protease and the cleavage products detected via lateral flow-based methods/chemistries.

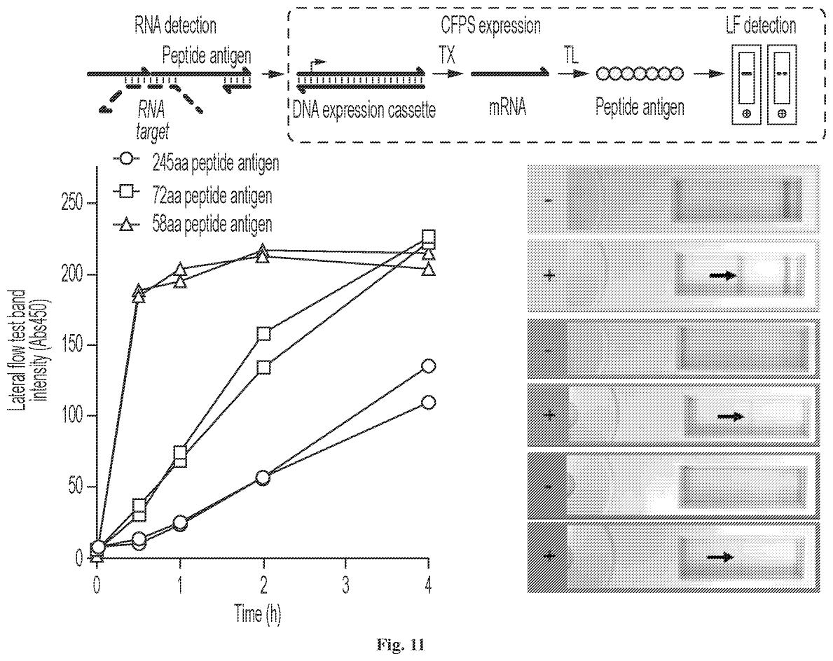

Example 8: This example FIGS. 10A and 10B show the production of various fragment sizes of the same peptide antigen output. Six different dsDNA fragments encoding pieces of the HIV p24 peptide antigen were incubated to generate the following peptide antigen fragments:

A.
AISPRTLNAWVKVVEEKANPDCKTILKALGPAATLEEMMTACQGV
(4.8 kD)
B.
PEVIPMFSALSEGATPQDLNTMLNTVGGHQNPDCKTILKALGPAATLEE
MMTACQGV (6.0 kD)
C.
AAMQMLKETINEEAAEWDRVHPVHAGNPDCKTILKALGPAATLEEMMTA
CQGV (5.7 kD)
D.
GQMREPRGSDIAGTTSTLQEQIGWMTNNPPIPNPDCKTILKALGPAATL
EEMMTACQGV (6.3 kD)
E.
LNKIVRMYSPTSILDIRQGPKEPFNPDCKTILKALGPAATLEEMMTACQ
GV (5.6 kD)
F.
YVDRFYKTLRAEQASQEVKNWMTETLLVQNANPDCKTILKALGPAATLE
EMMTACQGV (6.5 kD)

The reaction product was loaded with the lateral flow strips. Results indicated that different fragments of the same peptide antigen output can be detected using lateral flow. The WT (untruncated) version of the p24 output was used (WT=25.5 kD), as well as a no temple control.

Example 9: This example shows that peptides that are smaller in size are detected more rapidly than larger peptide outputs. Peptide antigens of three different sizes (p24 245aa, p24 fragment 72aa, and CARD18 58aa) were incubated with a cell-free protein synthesis (CFPS) composition at room temperature for 0-4 hours to allow the transcription and translation of the peptide antigen reporter. Results showed (FIG. 11) that the reporter of 58aa was generated at a faster rate and with a stronger band intensity, followed by the reporter of 72aa, and lastly the reporter of 245 aa. The 58 aa reporter had a significantly higher detection signal 30 minutes into the reaction compared to the other two larger reporters.

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

We claim:

1. A system comprising:
a plurality of nucleic acid molecules having different nucleotide sequences;
a set of ligation oligonucleotides, comprising:
a first ligation oligonucleotide whose nucleotide sequence includes a templating element and a first target hybridization element; and
a second ligation oligonucleotide whose nucleotide sequence includes a second target hybridization element and optionally a second templating element; and
optionally one or more bridging oligonucleotides whose nucleotide sequence is or comprises one or more additional target hybridization elements,
wherein the target hybridization elements bind to different portions of a common target site, so that, when the plurality of nucleic acid molecules includes at least one nucleic acid molecule whose nucleotide sequence includes the target site, then the set of ligation oligonucleotides hybridizes to the target site and forms a gapped nucleic acid strand susceptible to ligation with a ligase to generate a ligated strand that is or generates a product amenable to lateral flow assessment.


SEQUENCE LISTING (6 sequences)

SEQ ID NO: 1 - protein, 45 aa, Human immunodeficiency virus
AISPRTLNAWVKVVEEKANPDCKTILKALGPAATLEEMMTACQGV

SEQ ID NO: 2 - protein, 57 aa, Human immunodeficiency virus
PEVIPMFSALSEGATPQDLNTMLNTVGGHQNPDCKTILKALGPAATLEEMMTACQGV

SEQ ID NO: 3 - protein, 53 aa, Human immunodeficiency virus
AAMQMLKETINEEAAEWDRVHPVHAGNPDCKTILKALGPAATLEEMMTACQGV

SEQ ID NO: 4 - protein, 59 aa, Human immunodeficiency virus
GQMREPRGSDIAGTTSTLQEQIGWMTNNPPIPNPDCKTILKALGPAATLEEMMTACQGV

SEQ ID NO: 5 - protein, 51 aa, Human immunodeficiency virus
LNKIVRMYSPTSILDIRQGPKEPFNPDCKTILKALGPAATLEEMMTACQGV

SEQ ID NO: 6 - protein, 58 aa, Human immunodeficiency virus
YVDRFYKTLRAEQASQEVKNWMTETLLVQNANPDCKTILKALGPAATLEEMMTACQGV